SEQUENCE LISTING (3 sequences)

SEQ ID NO: 1 - DNA, 693 bp, Influenza virus
atggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcactctcggtctggacatcgagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttga

SEQ ID NO: 2 - DNA, 1479 bp, Rotavirus sp.
atggcaacctttaaggatgcttgttttcattatagaaggattacaaaactaaacagggaattactgagaattggagcaaactcagtatggactccagtttcttcaaataaaattagaggatggtgcattgagtgttgtcaattgactgaattgaccttttgtcatggatgctcattggctcacgtttgtcaatggtgcattcaaaacaaacgttgcttcttagacaatgaaccacatcttttaaagttaagaacctttgaatctccaataacaaaggaaaaattgcagtgcattattgatttatataatctactatttccaatcagccctggtatcataaatagatttaaaaaagcagttaaacaaagaaagtgtagaaatgaaagtgataaatcgtggtataatcaattactacttccaattactctaaatgccgcagtttttaaatttcattcgagggaaatttacatttttggattttatgaaggatcatcagcatgcatagatttaccatatagacttgtaaattgcattgacttatatgataaattgctgttagatcaaataaattttgaaagaatgagttgtcttccagataagttacaatctatctatgcaaataattattttaaattgagcagacttccttcaatgaagttgaaacaagtttattattcagacttctccaaacaaaatttgattaataagtataaaactaagagtcgcatagtacttagaaatctgactgaattcacttgggattctcaaattgatttgcatcatgatctaatcaataacaaagataaaatacttgcagcattatcaacatcatcgttaaaacaattcgaaacacatgatttaaatttaggaagagtaaaagctgacatttttgaacttggacatcattgcaaaccaaattatatttcatcaaatcattggcaaccggcatcaaaaattttccagtgtaagtggtgcaatgtaaagtatgcatttagagatatggattggaagatggaatcaatgtataatgaactcttaagttttctacagtcctgttacaaaagtaatgttaatgtagggcattgtagttcaattgaaagtgtttatccactagtcaaagatatgctttggcattcaattacaaaatatattgatcaaactattgagaaattatttgatgcaatgaatccggtgcaagtgaatgagcaacttgtaattaacttctattggcaaatagatatcgctctgtacactcacatcaaaatgatactaaaaactgaagctcttccgtttactttcacacttaaccagttcaattctataattaagggaatcattaaccaatggtgtgacgtcgctgaattggatcttttaccgttatgcactgaacaaaccgacaaattggttaaactgaaagaagaagggaaactgtctgaagagtatgagctcctaatctcggattccgaagacgacgactga

SEQ ID NO: 3 - DNA, 158 bp, Adenovirus
ggctcgactccgtggcctggaggctaagcgaacgggttgggctgcgcgtgtaccccggttcgaatctcgaatcaggctggagccgcagctaacgtggtactggcactcccgtctcgacccaggcctgcacaaaacctccaggatacggaggcgggtcg

(19) United States
(12) Patent Application Publication
(10) Pub. No.: US 2011/0117124 A1
(43) Pub. Date: May 19, 2011

(54) ENHANCEMENT OF TRANSGENE EXPRESSION FROM VIRAL-BASED VACCINE VECTORS BY EXPRESSION OF SUPPRESSORS OF THE TYPE I INTERFERON RESPONSE

(76) Inventors: Jerald C. Sadoff, Washington, DC (US); John Fulkerson, Silver Spring, MD (US); Mohamad Fakruddin-Jamiluddin, Frederick, MD (US); Michele R. Stone, Brookeville, MD (US); Ravi Anatha, Gaithersburg, MD (US)

(21) Appl. No.: 12/675,535

(22) PCT Filed: Aug. 29, 2008
(86) PCT No.: PCT/US08/74758

§ 371 Date: Nov. 19, 2010

Related U.S. Application Data
(60) Provisional application No. 60/969,283, filed on Aug. 31, 2007.

(51) Int. Cl. A61K 39/04; C12N 15/63; A61K 39/002; A61K 35/76; C12N 15/86; A61P 31/06; A61P 33/06; A61P 35/00; A61P 37/04

(52) U.S. Cl. 424/199.1; 435/320.1; 424/93.2; 435/456

(57) ABSTRACT

Viral-based vectors are genetically engineered to express inhibitors of the anti-viral immune system (e.g. inhibitors of the type I interferon response) in order to enhance transgene expression. The transgenes may encode antigens or other therapeutic agents.

### BACKGROUND OF THE INVENTION

1. Field of the Invention

The invention generally relates to the enhancement of transgene expression from viral-based vectors. In particular, the invention provides viral-based vectors that encode genetically engineered inhibitors of the type I interferon response, together with genes of interest that provide host cell active responses (e.g. immunostimulatory, therapeutic, or selectively apoptotic) thereby enhancing transgene expression.

2. Background of the Invention

The innate immune system serves as a first line of defense system against invading pathogens, including bacteria or viruses. Eukaryotic cells possess the inherent capability to recognize components of viruses and microbes via a number of cell surface and intracellular geermline-encoded pattern-recognition receptors (PRRs) such as the Toll-like receptors (TLRs), the Nod-like (nucleotide-binding oligomerization domain) receptors, and the RNA helicases RIG-I (retinoic acid-inducible gene-I) and MDA5 (melanoma differentiation associated gene 5). Binding of viral or bacterial components by these receptors mediates up-regulation and production of antibacterial and antiviral effectors. Jawed vertebrates which evolved an adaptive immune system also developed the interferon cytokine family that is dedicated to autocrine and paracrine signaling of the presence of infection and facilitates communication among cells that provide protection against infectious agents, including viruses and intracellular bacteria. Similarly, they may activate mechanisms within an infected cell intended to limit the infection by interruption of cellular processes or degradation of foreign material. Interferon (IFN)-α and -β comprise the type I IFN family and were first identified as humoral factors that confer an antiviral state on cells. Among the autocrine IFN-induced effector and modulator proteins essential for the antiviral actions of type I IFNs are the RNA-dependent protein kinase (PKR), the 2,5′-oligoadenylate synthetase (OAS), RNase L, and the Mx protein GTPases. Double-stranded or highly structured RNA plays a central role in modulating protein phosphorylation and RNA degradation catalyzed by the TN-inducible PKR kinase which halts RNA translation and the OAS-dependent RNase L which degrades RNA, respectively, and also in RNA editing by the IFN-inducible RNA-specific adenosine deaminase (ADAR1). The expression of IFN-α/β is effectively controlled by transcription factors of the IFN regulatory factor (IRF) family. For example, double-stranded RNA and lipopolysaccharide, when recognized by TLR3 and TLR4 respectively, lead to IRF-3 and IRF-7 activation; TLR7 and TLR9 detect single-stranded RNA and CpG DNA and stimulate IRF-5 and IRF-7 via a MyD88-dependent pathway also involving IRAK1/4 and TRAF6.

Most successful viral pathogens of mammals have evolved mechanisms of blocking these autocrine and paracrine responses, enabling them to establish infection. Moreover, certain viruses or double-stranded RNA activate TLR-independent PRR responses, which signal via the cytosolic RNA helicases RIG-I and/or MDA5 through the adapter molecule IPS-1 (interferon-promoter stimulator 1) thereby stimulating IRF-3 and IRF-7 dependent transcription of specific response genes. Examples of viral proteins evolved to overcome this response include, but are not limited to, the NSP1 protein of rotavirus which binds IRF-3 and prevents nuclear translocation, the C12R protein of ectromelia virus which binds IFN-α/β, NS1 of influenza which prevents nuclear translocation of IRF-3 and interferes with RIG-1 dependent signaling, and the NS3/4A protease of hepatitis C virus which specifically cleaves the MAV and TRIF proteins involved in signaling the transcription of IFN-α/β.

Vaccines have been successful in eradicating or reducing the occurrence of many diseases. However, some diseases have thus far proven to be recalcitrant to immunization efforts. For others, the vaccines currently in use are not optimally effective and/or have untoward side effects. Thus, the need for new approaches to the vaccine design is ongoing. The use of vectors based on attenuated viruses that are naturally capable of infecting eukaryotic cells is particularly promising. Such vectors can be readily genetically engineered to contain and express transgenes encoding antigens of interest. Unfortunately, in many instances the administration of such vectors does not result in the production of sufficient antigen to elicit a protective immune response in the recipient. This is often because the host cells that are infected by the vaccine vector do not distinguish between viral infectious agents and attenuated viral vaccine vectors. The host cell reacts to a vaccine viral vector as it would to a true infectious agent: the cell mounts an immune response, especially an IFN I response, which destroys or attenuates the ability of the vaccine vector to produce the encoded antigens, thereby defeating the purpose of the vaccine administration.

There is an ongoing need to develop new and improve existing vaccine delivery vehicles, and especially to solve the problem of host interference with the production of antigens by viral-based vaccine vectors.

### SUMMARY OF THE INVENTION

The present invention provides viral vectors which, in addition to being genetically engineered to contain nucleic acid sequences encoding host cell active amino acid sequences (sometimes referred to herein as “encoded factors”, and which can be one or more proteins or peptides of interest including enzymes, antigens, antibodies, therapeutic agents, apoptotic agents (e.g., TNF), cancer or tumor killing agents, etc.), they are also genetically engineered to contain nucleic acid sequences encoding factors that inhibit the mammalian anti-viral immune response (sometimes referred to herein as “suppressor factors” or “interfering factors”). For example, the encoded factors in the viral vector may be one or more antigens specific for tuberculosis or other diseases (malaria, human immunodeficiency, influenza, dengue, etc.). The vector will also be genetically engineered to encode suppressor factors that inhibit the mammalian IFN I response to viruses. As a result, the antigens that are encoded by the vector are transcribed and translated in eukaryotic host cells without interference or with diminished interference by the host cell's anti-viral immune response. Hence, the ability to produce an immunostimulatory response in a human or other mammal is increased. The invention also contemplates applications for enhanced delivery of proteins of interest (e.g., insulin, tumor necrosis factor, etc.) using the viral vectors since there will be either no interference or diminished interference by the host cell's anti-viral immune response.

It is an object of this invention to provide a recombinant viral vector, comprising one or more genetically engineered nucleic acids coding for a host cell type 1 interferon (IFN) response suppressor factor; and one or more genetically engineered nucleic acids coding for one or more host cell active amino acid sequences. The one or more genetically engineered nucleic acids coding for the one or more host cell active amino acid sequences are over expressed in the host cell. In one embodiment of the invention, the host cell type 1 IFN response interfering factor is rotavirus NSP1 or influenza virus NS1. In another embodiment, the host cell type IFN response interfering factor is rotavirus NSP1, influenza virus NS1, ectromelia virus C12R protein, hepatitis C virus NS3/4A protease, vaccinia virus vIFN-α/β Rc protein, adenovirus EIA protein, C proteins of paramyxovirus, or human papillomavirus (HPV) E6 oncoprotein. In yet another embodiment, the recombinant viral vector is derived from adenoviruses, baculoviruses, pox viruses, measles viruses, polioviruses, lentiviruses, hepatitis viruses, arboviruses or vesicular stomatitis viruses (or a wide variety of other viruses). In some embodiments, the encoded factors include one or more immunostimulatory amino acid sequences that are derived from one or more of rotavirus, influenza virus, ectromelia virus, hepatitis viruses (e.g., C, etc.), vaccinia virus, adenovirus, paramyxovirus, HPV, HIV, HTLV, enteroviruses, herpesviruses, EEE, VEE, West Nile virus, Norwalk virus, parvoviruses, dengue virus, and hemorrhagic fever virus (or a wide array of other viruses). In some embodiments, the one or more host cell active amino acid sequences are antigens, such as a Mycobacterium tuberculosis antigen. The encoded factors may also be enzymes, therapeutic peptides or proteins, apoptotic or anticancer agents, etc., where the nature of the encoded factors will depend on the application.

It is another object of this invention to provide a method of using a recombinant viral vector, comprising one or more genetically engineered nucleic acids coding for a host cell type 1 IFN response suppressor factor; and one or more genetically engineered nucleic acids coding for one or more host cell active amino acid sequences, to provide to a cell, in vitro or in vivo (e.g., in a mammal such as a human) the one or more host cell active amino acid sequences. On infecting the cell with the recombinant viral vector, greater production of the amino acids will result because the cell will have either no ability or a diminished ability to mount an effective mammalian IFN I response to the recombinant viruses. The one or more genetically engineered nucleic acids coding for the one or more host cell active amino acid sequences are over expressed in the host cell. In one embodiment of the invention, the host cell type 1 IFN response interfering factor is rotavirus NSP1 or influenza virus NS1. In another embodiment, the host cell type IFN response interfering factor is rotavirus NSP1, influenza virus NSI, ectromelia virus C12R protein, hepatitis C virus NS3/4A protease, vaccinia virus vIFN-α/β Rc protein, adenovirus E1A protein, C proteins of paramyxovirus, or human papillomavirus (HPV) E6 oncoprotein. In yet another embodiment, the recombinant viral vector is derived from adenoviruses, baculoviruses, pox viruses, measles viruses, polioviruses, lentiviruses, hepatitis viruses, arboviruses or vesicular stomatitis viruses (or a wide array of other viruses). In yet another embodiment, the encoded factors include one or more immunostimulatory amino acid sequences that are derived from one or more of rotavirus, influenza virus, ectromelia virus, hepatitis viruses (e.g., C, etc.), vaccinia virus, adenovirus, paramyxovirus, HPV, HIV, HTLV, enteroviruses, herpesviruses, EEE, VEE, West Nile virus, Norwalk virus, parvoviruses, dengue virus, and hemorrhagic fever virus (or a wide array of other viruses). In some embodiments, the one or more host cell active amino acid sequences are antigens, such as a Mycobacterium tuberculosis antigen. The encoded factors may also be enzymes, therapeutic peptides or proteins, apoptotic or anticancer agents, etc., where the nature of the encoded factors will depend on the application.

It is yet another object of the invention to provide a mechanism for tailoring a response in a host cell, in vitro or in vivo, to selectively provide greater or lesser amounts of type 1 IFN response. By selecting from amongst different type 1 IFN suppressor factors, a recombinant viral vector with a suppressor factor; and one or more genetically engineered nucleic acids coding for one or more host cell active amino acid sequences, can be provided. The tailored recombinant viral vector can provide to a cell, in vitro or in vivo (e.g., in a mammal such as a human) the one or more host cell active amino acid sequences in greater or lesser amounts depending on the interfering factor which is genetically engineered into the viral vector. Tailoring can also be achieved by selecting among different promoters, providing additional copies of nucleic acids coding for proteins of interest, and by other means. On infecting the cell with the recombinant viral vector, greater production of the amino acids will result because the cell will have either no ability or a diminished ability to mount an effective mammalian IFN I response to the recombinant viruses. The amount of the increased production can be tempered by the tailoring used to make the recombinant viral vector, such that in some applications significantly higher production can be achieved, while in other applications only slightly higher production is achieved. The tailoring contemplated herein contemplates the full spectrum from low to high production of the encoded factors in the host cell. The one or more genetically engineered nucleic acids coding for the one or more host cell active amino acid sequences are over expressed in the host cell to a degree controlled by the tailoring employed. In one embodiment of the invention, the host cell type 1 IFN response interfering factor is rotavirus NSP1 or influenza virus NS1. In another embodiment, the host cell type IFN response interfering factor is rotavirus NSP1, influenza virus NS1, ectromelia virus C12R protein, hepatitis C virus NS3/4A protease, vaccinia virus vIFN-α/β Rc protein, adenovirus E1A protein, C proteins of paramyxovirus, or human papillomavirus (HPV) E6 oncoprotein. In yet another embodiment, the recombinant viral vector is derived from adenoviruses, baculoviruses, pox viruses, measles viruses, polioviruses, lentiviruses, hepatitis viruses, arboviruses or vesicular stomatitis viruses or a wide array of other viruses. In yet another embodiment, the encoded factors include one or more immunostimulatory amino acid sequences that are derived from one or more of rotavirus, influenza virus, ectromelia virus, hepatitis virus (e.g., C, etc.), vaccinia virus, adenovirus, paramyxovirus, HPV, HIV, HTLV, enteroviruses, herpesviruses, EEE, VEE, West Nile virus, Norwalk virus, parvoviruses, dengue virus, and hemorrhagic fever virus (or a wide array of other viruses). In some embodiments, the one or more host cell active amino acid sequences are antigens, such as a Mycobacterium tuberculosis antigen. The encoded factors may also be enzymes, therapeutic peptides or proteins, apoptotic or anticancer agents, etc., where the nature of the encoded factors will depend on the application.

### BRIEF DESCRIPTION OF THE DRAWINGS

FIG. 1A-C. Sequences of anti-viral immune response inhibitors. A, DNA sequence of NS1 from influenza virus (SEQ ID NO: 1); B, DNA sequence of NSP1 from rotavirus (SEQ ID NO: 2); C, RNA gene sequence of VAI from adenovirus (SEQ ID NO: 3)

FIG. 2. IFN antagonist gene VAI increases the expression of TB.S antigen. Immunoblot depicting the enhanced production of adenovirus expressed TB.S antigen from VAI gene transfected HeLa cells (lane 2 and 4) compared to untransfected HeLa cells (lanes 1 and 3). Higher expression of TB.S antigen was seen at both 50 and 100 MOIs. There was no expression of TB.S antigen observed in VAI transfected (lane 6) and untransfected (lane 5) HeLa cells infected with empty vector Ad35. Lane 7 and 8 shows uninfected control HeLa cells untransfected and transfected with VAI gene, respectively.

### DETAILED DESCRIPTION

The present invention is based on the development of viral vectors which, in addition to being genetically engineered to contain nucleic acid sequences encoding one or more amino acid sequences (e.g., proteins, peptides, enzymes, or other encoded factors or agents) of interest that are active within a host cell, are also genetically engineered to contain nucleic acid sequences encoding one or more factors that inhibit the host cell immune response against viruses (referred to as “suppressor factors” or “interfering factors” herein). Consequently, mammalian host cells infected with the viral vaccine vectors produce the protein(s) of interest without being encumbered or inhibited by the host cell's anti-viral immune response. The amino acids are expressed from the genetically engineered nucleic acids and are host cell active, i.e. they posses an activity (property, characteristic, etc.) that is beneficial to the host cell. In some embodiments, the amino acids are antigens, and increased expression of these antigens in cells in which immune response inhibitors are also expressed from the vector leads to improved cellular and humoral immune responses to the antigens. In addition, the improvements in transgene expression may also enable a reduction in the vector dosage that is required to achieve an adequate immune response. In other embodiments, the encoded factors may be otherwise therapeutic in nature. Herein, the term “therapeutic” is used to denote factors that are not antigens, but which have some other beneficial effect within the host cell (for example, insuliin, Factor VIII and other peptides are “therapeutic”). The encoded factors may also be selectively apoptotic (e.g., TNF) and may function as anticancer agents.

If the interfering factors (usually proteins) that inhibit the host cell's immune response are viral in origin, they may be heterologous (i.e. derived from or originating from a virus type that differs from the viral vaccine vector) or homologous (i.e. derived from or originating from the same type of virus). For example, an adenoviral vector may be genetically engineered to contain and express an immune system inhibitor from, for example, a heterologous virus such as influenza virus. Alternatively, an adenoviral vector may be genetically engineered to contain and overexpress a homologous adenoviral immune system inhibitor, even though the viral vector may already naturally encode the inhibitor. In the latter case, the homologous immune system inhibitor is overexpressed in the genetically engineered vector at levels above a normal or characteristic level of expression in the virus of origin, or in the viral vector prior to being genetically engineered according to the invention. This is accomplished, for example, by genetically engineering the viral vector to contain multiple copies of a gene encoding the inhibitor, or by genetically engineering the viral vector so that transcription of the inhibitor is driven by a more effective promoter (e.g. a super promoter), or by combinations of these strategies, etc.

A further aspect of the invention is the provision of “tuned” or “tunable” viral vectors in which the amount or activity of an immune system inhibiting factor within a recipient host cell is tailored to a desired level. Such tuning may be carried out by, for example, manipulating or varying the identity and/or expression pattern of the one or more factors that inhibit the immune response. For example, the promoter that drives transcription of a factor or of a protein of interest can be selected so that a desired level of transcription is attained, with very active promoters being used if high levels of transcription are desired, and weak promoters being used if low levels of transcription are desired. Further, particular pathways of the immune response may be specifically targeted by differential expression of factors which narrowly or specifically inhibit the selected pathways. For example, the NS1 protein of influenza A prevents the nuclear translocation of IRF-3 and prevents RIG-1 dependent signaling early in infection, while the NS3/4A protease of hepatitis C virus cleaves factors directly involved in the translation of IFN-α/β later in the response by an unrelated mechanism and the C12R protein of ectromelia virus binds and inactivates IFN-α/β produced later in response to infection by yet another mechanism. By selectively choosing the particular promoters that are used within a construct, and by selectively choosing the particular factors or combinations of factors that are encoded by a construct, a tailored host cell response can be elicited. Other ways of varying the expression of the factors include but are not limited to: the use of inducible promoters; the use of cell- or tissue-specific promoters; inclusion of various genetic elements that increase or decrease transcription (e.g. enhancer sequences, etc.); the use of preferential or non-preferred codon sequences; etc. In addition, the factors or host cell active proteins of interest may be altered or selected so as to possess increased or decreased activity, depending on the goal of vector administration.

By “inhibiting” an immune response we mean that the typical or normal immune response that is elicited by the presence of a virus within a eukaryotic cell, is fully or partially inhibited, lessened, decreased, impeded, etc. Such inhibition may be detected and measured in any of several ways that will occur to those of skill in the art, including but not limited to: detection of a decrease in an amount, activity or attribute of a substance that is a hallmark of, is characteristic of or is associated with the anti-viral immune response (e.g. IFNα, IFNβ, etc.). The level of inhibition is generally at least about 25%, preferably about 50% and more preferably about 60, 70, 80, 90 or 100%. A level of inhibition is typically measured by detecting a difference between an amount of one or more substances produced in a host cell that has been transfected with a viral vector of the invention (a vector that encodes one or more transgenes plus one or more immune system inhibitors), compared to the amount of the same substance produced in a control cell (a cell transfected with a viral vector that encodes the one or more transgenes but does not encode an immune system inhibitor).

Similarly, “enhancing” expression of a transgene generally refers to an increase, augmentation, etc. in an amount of a transgene that is expressed (i.e. transcribed and translated) within a host cell transfected with a vector of the invention, compared to a control cell. Such enhancement may be measured by any of several methods that will occur to those of skill in the art, e.g. by detection of an increase in an amount, activity or attribute of a transgene product that is produced from the vector; by detection of an increase in an amount, activity or attribute of a substance associated with the transgene product that is produced (e.g. mRNA, substance or effect produced by the transgene product, antibodies to the transgene product, etc.). The level of enhancement is generally at least about 25%, preferably about 50% and more preferably about 60, 70, 80, 90 or 100%, or even more.

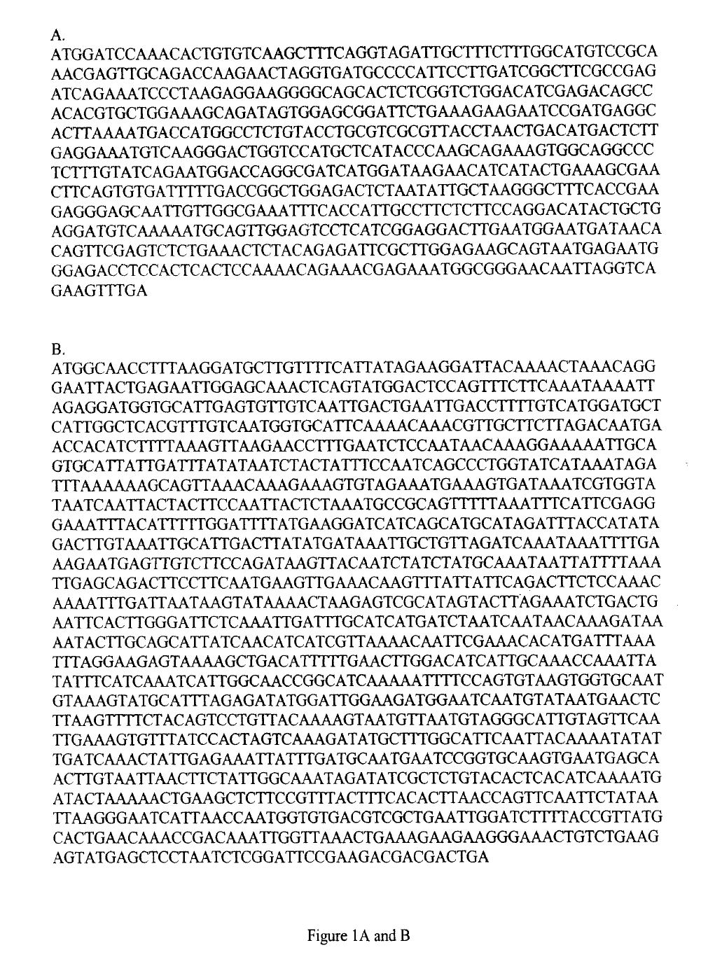

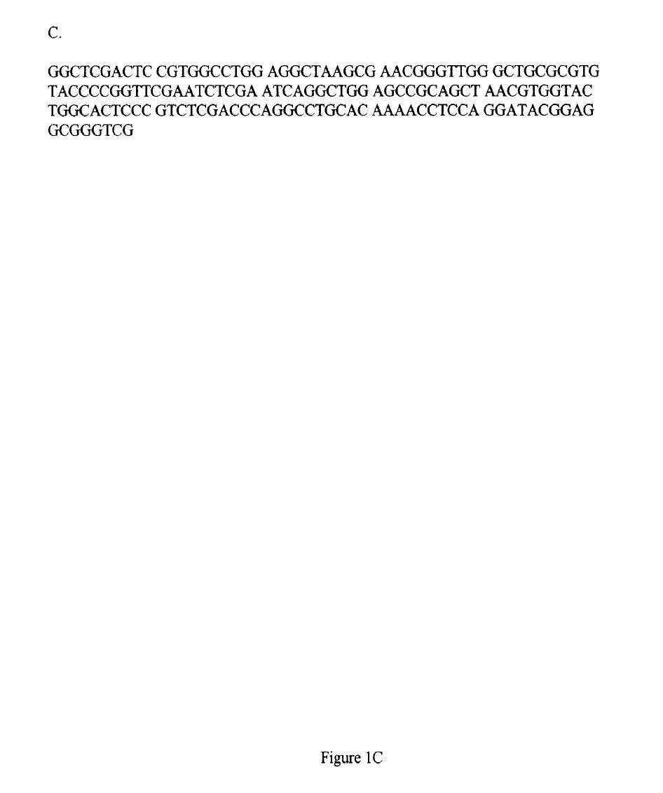

The fundamental importance of the IFN system as a host defense against viral infection is further illustrated by the finding that a number of viruses encode gene products that antagonize the IFN-induced antiviral response. Viruses utilize several different strategies to block the induction and action of IFN-inducible proteins. Both DNA and RNA viruses encode proteins that impair the activity of the IFN signaling pathway. Multiple mechanisms appear to be involved. Among these is mimicry. Several examples exist in which viruses encode products that mimic cellular components of the IFN signal transduction pathway. This molecular mimicry can lead to an antagonism of the IFN signaling process. Poxviruses, for example, encode soluble IFN receptor homologues (vIFN-Rc). These vIFN-Rc homologues are secreted from poxvirus-infected cells and bind IFNs, thereby preventing them from acting through their natural receptors to elicit an antiviral response. A vIFN-α/βRc protein is secreted by vaccinia virus and several additional orthopoxviruses. The vIFN-α/β receptor homologue, the B18R gene product in the Western Reserve strain and the B19R product in the Copenhagen strain, binds several different IFN-αsubspecies as well as IFN-β and blocks IFN-α/β signaling activity. Three additional DNA viruses that affect IFN signaling are adenovirus, papillomavirus, and human herpesvirus 8 (HHV-8). The adenovirus E1A protein blocks IFN-mediated signaling at a point upstream of the activation of ISGF-3. The DNA binding activity of ISGF-3 is inhibited by E1A. The C proteins of SeV (SeV), a paramyxovirus that replicates in the cytoplasm of the host, circumvents the IFN-induced antiviral response by interfering with the transcriptional activation of IFN-inducible cellular genes. In the case of Sendai virus, the C proteins interfere with IFN action in at least two ways. C proteins prevent the synthesis of STAT-1 and they also induce an increased turnover of STAT-1. Human papillomavirus (HPV) E6 oncoprotein binds selectively to IRF-3 but only very poorly to other cellular IRFs including IRF-2 and IRF-9. Association of E6 with IRF-3 inhibits transactivation, thereby providing HPV with a mechanism to circumvent the IFN response. Adenovirus E1A protein also inhibits IRF-3-mediated transcriptional activation by a mechanism dependent on the ability of E1A to bind p300. HHV-8, a gamma herpes virus associated with Kaposi's sarcoma, synthesizes an IRF homologue (vIRF) that functions as a repressor of transcriptional activation induced by IFN-03. The HHV-8-encoded vIRF protein also represses IRF-1-mediated transcriptional activation. Two other herpesviruses, varicella-zoster virus (VZV) and cytomegalovirus (CMV), also disrupt the function of the IFN signal transduction pathway. VZV inhibits the expression of STAT-1 and JAK-2 proteins but has little effect on JAK-1. A different strategy of antagonism occurs in CMV-infected cells, where MHC class II expression also is inhibited. There is a specific decrease in the level of JAK-1 due to enhanced protein degradation in CMV-infected fibroblasts. Several nonsegmented negative-strand RNA viruses encode gene products that antagonize IFN receptor-mediated signaling from type I IFN receptors. For example, infection with simian virus 5 or mumps virus leads to an increased proteosome-mediated degradation of STAT-1 whereas in cells infected with parainfluenza virus type 2 there is a degradation of STAT-2. The VP35 protein of Ebola virus, a negative-strand RNA virus, functions as a type I IFN antagonist although the precise biochemical mechanism of the antagonism has not yet been defined. VP35 inhibits virus induction of the IFN-β promoter and dsRNA- and virus-mediated activation of ISRE-driven gene expression. Nucleic acid sequences encoding three exemplary inhibitors (NS1 from influenza virus, NSP1 from rotavirus, and VAI from adenovirus) are presented in FIGS. 1A-C.

IFN suppressing factors may also be obtained from other non-viral sources, for example, from the host cell (e.g. suppressors of cytokine signaling (SOCS), dominant negative of PKR and dominant negative of RNaseL) and may be utilized in the practice of the present invention. Any factor that suppresses or attenuates the IFN response (e.g. siRNAs against Interferon stimulated genes) and which is encoded by a nucleic acid sequence that can be genetically engineered into and successfully expressed from a viral expression vector may be used in the practice of the present invention. Examples include but are not limited to those described above, as well as various autocrine IFN-induced effector and modulator proteins essential for the antiviral actions of type I IFNs such as RNA-dependent protein kinase (PKR); 2,5′-oligoadenylate synthetase (OAS); RNase L; Mx protein GTPases; IFN-inducible RNA-specific adenosine deaminase (ADAR1); IFN regulatory factors such as IRF-5 and IRF-7; transcription factors of the (IRF) family such as TLR3, TLR4, TLR7 and TLR9; factors such as IRAK1/4 and TRAF6; RLR, MyD88, TAK1, TOLLIP, TIFA, etc.

One or more functional forms of such factors are operably encoded by the viral vectors of the invention. By “functional form” we mean that the factor that is encoded possesses as least about 25%, preferably about 50%, and more preferably about 100% or more of its usual activity when transcribed from a viral vector of the invention in a suitable host cell, e.g. a mammalian host cell. By “operably encoded” we mean that the nucleic acid sequences encoding the factor are amenable to successfully transcription and translation within a suitable host cell, such as a mammalian cell.

The vectors of the invention are viral-based vectors. By “viral-based” we mean vectors or vehicles derived from or based on naturally occurring viruses. Such vectors generally will have been changed from their natural form via genetic engineering in any of several possible beneficial ways. For examples, the viral vectors are generally attenuated so that they do not cause disease symptoms or cause only mild disease symptoms; they may be altered so as to be incapable of replication within a host cell; they may be genetically engineered to contain nucleic acid sequences that facilitate the introduction of heterologous genes (e.g. passenger gene or transgenes) from other organisms, or multiple copies of genes from like organisms; they may encode deletions in genes which activate complement, IRES from viruses, leader peptide sequences, sequences designed to increase mRNA stability, etc.

In particular for the purposes of the present invention, the viral vectors are genetically engineered to contain and express 1) one or more proteins or polypeptides of interest and 2) one or more factors that inhibit the immune response of mammalian cells to invasion by viruses. Further, the identity and expression patterns of the one or more polypeptides and the one or more factors that inhibit the immune response can be designed so as to cause a tailored or tuned response within the host cell.

Generally, in the practice of the invention, the viral-based vectors are non-replicating, have limited replication in target cells/tissues, and/or the factors that inhibit the host cell's anti-viral response are expressed in a cell- or tissue-specific manner. This is because repression of the host cell's ability to ward off viral infection should be only temporary or confined to a specific location in the host, or should selectively down-regulate only certain triggered mechanisms, to avoid making the host generally susceptible to infection by viruses. For example, adenovirus-based vectors are typically E1-E3 deleted and carry transgenes under the transcriptional control of a CMV or other viral/mammalian promoter. The E1-E3 gene products are normally involved in transcription of viral DNA, the rearrangement of the target cell cytoskeleton, down regulation of MHC and the assembly of new virions. While inclusion of a suppressor of the Type I IFN response may enhance the translation, and in some cases perhaps the transcription of both vector and passenger gene sequences, it does not offer a mechanism for replacement of deleted genes or gene function. Further, as there are numerous mechanisms of activating the innate immune response by cell surface and intracellular pattern recognition, it is both difficult and undesirable to obviate the type I IFN response completely, particularly in the case of a vaccine vector. Also, while the IFN a/13 response has some local paracrine function, the main object of the invention is incorporation of suppressors which down regulate autocrine responses. Finally, the host immune response to intracellular pathogens is not limited to Type I IFNs nor are type I IFNs required for cognate immunity.

Examples of suitable virus-derived vectors include but are not limited to those derived from adenovirus (both replicating and non-replicating), baculoviral vectors, as well as vectors derived from pox viruses, measles viruses, polioviruses, influenza viruses, vesicular stomatitis virus, retroviruses, lentiviruses, etc. In addition, various nanoengineered substances (e.g. Ormosil) may be employed.

With respect to the transgenes that are encoded by the vector, in one embodiment of the invention, the transgenes encode antigens to which it is desired to elicit a cell or humoral immune response. Generally, such antigens are proteins, polypeptides or peptides, or antigenic fragments thereof, from a disease causing organism or agent such as a virus, bacteria, or eukaryotic parasite. The antigen may be a whole protein, or a portion of a protein (e.g. one or more antigenic regions thereof), or one or more antigenic epitopes from a protein. Generally, such epitopes are, for example, at least about 8 amino acids in length. Such viral vectors may be of any serotype, and may include antigens associated with diseases or etiologic agents such as influenza virus; Retroviruses, such as RSV, HTLV-1, and HTLV-II, Papillomaviridae such as HPV, Herpesviruses such as EBV, CMV or herpes simplex virus; Lentiviruses, such as HIV-1 and HIV-2; Rhabdoviruses, such as rabies; Picornoviruses, such as Poliovirus; Poxviruses, such as vaccinia; Rotavirus; and Parvoviruses, such as adeno-associated virus 1, Mycobacterium spp., Helicobacter pylori, Salmonella spp., Shigella spp., E. coli, Rickettsia spp., Listeria spp., Legionella pneumoniae, Pseudomonas spp., Vibrio spp., Bacillus anthracis, Borellia burgdorferi., Plasmodium spp., such as Plasmodium falciparum; Trypanosome spp. such as Trypanosoma cruzi; Giardia spp. such as Giardia intestinalis; Boophilus spp., Babesia spp. such as Babesia microti; Entamoeba spp. such as Entamoeba histolytica; Eimeria spp. such as Eimeria maxima; Leishmania spp.; Schistosome spp., Brugia spp., Fascida spp., Dirofilaria spp., Wuchereria spp., Onchocerea spp., etc. In particular, M. tuberculosis antigens such as Rv1733, Rv3130c, Rv2627c, Rv2628, Rv3641c, Rv3135, Rv3136, Rv0383c, Rv0394c, Rv3514, Rv3532, Rv1997, Rv0159c, Rv1039c, Rv1197, Rv3620c, Rv2347c, and Rv1792; and/or malaria antigens such as circumsporozoite protein (CSP) or peptides fragments thereof, may be utilized.

In addition, the vectors of the invention may be used to cause an immune reaction to cancer antigens, examples of which include but are not limited to muc1, survivin, ciliary neurotrophic factor, cyclooxygenase 1, fibroblast growth factor, endothelial differentiation factor, MAGE-1, tyrosinase, etc.

Further, more than one antigen may be encoded in the viral vector, either individually or as chimeric antigens, i.e. a single translatable gene product that comprises two or more different antigens. The antigens may be related to the same disease (e.g. several tuberculosis antigens may be encoded in a single, contiguous transcript) or may be related to different diseases (e.g. diphtheria, pertussis and tetanus antigens may be included in a single transcript). Alternatively, multiple antigens may be encoded separately using, for example, bicistronic expression employing internal ribosomal entry sites (IRES), multiple individual promoters and stop signals, or other similar devices for transcribing multiple encoded proteins, polypeptides or peptides. Further, the arrangement of the antigen and the factor that inhibits the anti-viral immune response may also be encoded separately, or as a chimera, or using a device for multiple transcription, etc.

In other embodiments of the invention, moieties other than antigens are encoded by the viral vectors. For example, various proteins/polypeptides/peptides with a beneficial action may be encoded, examples of which include but are not limited to proteins that are missing or which function improperly in an individual (e.g. insulin, CFTR, etc.). The methods of the invention can be used, for example, for the delivery of proteins for correction of hereditary disorders. Such genes would include, for example, replacement of defective genes such as the cystic fibrosis transmembrane conductance regulator (CFTR) gene for cystic fibrosis; or the introduction of new genes such as the integrase antisense gene for the treatment of HIV; or genes to enhance Type I T cell responses such as interleukin-27 (IL-27); or genes to modulate the expression of certain receptors, metabolites or hormones such as cholesterol and cholesterol receptors or insulin and insulin receptors; or genes encoding products that can kill cancer cells such as tumor necrosis factor (TNF)-related apoptosis-inducing ligand (TRAIL); or a naturally occurring protein osteoprotegerin (OPG) that inhibits bone resorption; or to efficiently express complete-length humanized antibodies, for example, humanized monoclonal antibody that acts on the HER2/neu (erbB2) receptor on cancer cells.

In some embodiments of the invention, the viral vectors are included in vaccine preparations and/or preparations for eliciting an immune response, or preparations for some other type of treatment of a mammal. The compositions of the invention include substantially purified viral vectors as described herein, and a pharmacologically suitable carrier. The preparation of such compositions is well known to those of skill in the art. Typically, such compositions are prepared either as liquid solutions or suspensions, however solid forms such as tablets, pills, powders and the like are also contemplated. Solid forms or concentrated forms suitable for mixing with, solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of viral vector in the formulations may vary, but generally will be from about 1-99%. The compositions may further comprise an additional adjuvant, suitable examples of which include but are not limited to Seppic, Quil A, aluminum based adjuvants such as Alhydrogel, etc. The compositions may contain a single type of viral vector, or more than one type of viral vector may be utilized in a preparation, i.e. the preparations may comprise a “cocktail” of such vectors.

The methods of the present invention involve administering a composition comprising one or more viral-based vectors as described herein in a pharmacologically acceptable carrier to a mammal. While the mammal will generally be a human, this need not always be the case. Veterinary applications of the invention are also contemplated. The preparations may be administered by any of the many suitable means which are well known to those of skill in the art, including but not limited to by injection, orally, intranasally, transcutaneously, intravenously, intraperitoneally, subcutaneously, intramuscularly, by inhalation, etc. In addition, the compositions may be administered alone or in combination with other medicaments or immunogenic compositions, e.g. as part of a multi-component vaccine. Further, administration may be a single event, or multiple booster doses may be administered at various timed intervals, e.g. in the case of vaccines, to augment the immune response; or in the case of treating other diseases such as cancer, to eliminate cancer cells that escaped a first round of treatment; or for any other reason. Administration is preferably prophylactic i.e. before exposure to a disease-causing agent has occurred, or is suspected to have occurred. However, administration may also be after the fact, i.e. after a known or suspected exposure to a disease causing organism, or therapeutically, e.g. after the appearance of disease symptoms.

The amount of the viral vector to be administered may vary depending on characteristics of the subject to whom it is administered (for example, the species, gender, age, genetic makeup, general health, etc.), as well as the disease or condition that is being treated. Generally, the dosage employed may be about 10³ to 10¹¹ viable organisms, preferably about 10³ to 10⁹ viable virus particles (or pfu), as described (Shata et al., Vaccine 20:623-629 (2001); Shata and Hone, J. Virol. 75:9665-9670 (2001)).

The invention also provides methods of increasing production of a protein, polypeptide or peptide in an individual in need thereof. The method includes co-expressing, from a viral vector, the protein/polypeptide/peptide with a factor that inhibits an anti-viral immune response in cells of the individual. If the protein/polypeptide/peptide is an antigen associated with a disease causing agent, then the method may be a method of inducing an immune response in the individual. If the immune response that is elicited is protective (i.e. if the immune response prevents or lessens the occurrence of disease symptoms caused by the disease-causing agent), then the method may also be referred to as a method of vaccinating the individual. Alternatively, the invention also provides methods of treating a disease or lessening symptoms of a disease by administering a viral vector of the invention to an individual. In this case, the viral vector transgene encodes a protein/polypeptide/peptide that is necessary to or helpful in preventing or lessening symptoms of disease in the individual to whom the viral vector is administered.

### EXAMPLE

### Example 1

### IFN Antagonist Gene VAI Increases the Expression of TB.S Antigen

This experiment describes the use of an interferon antagonist gene to reduce the negative effects of IFNs on the expression of tuberculosis antigens cloned in an adenoviral vector. Experiments were designed and conducted as follows.

Ad35-TB.S is a replication deficient, E1 deleted derivative of adenovirus 35 which encodes a fusion protein of antigens 85A, 85B and TB 10.4 from M. tuberculosis. As with other group B adenoviruses, adenovirus 35 infects mammalian cells expressing the surface marker CD46 and thus is capable of infecting the vast majority of all nucleated human cells. The interferon antagonist gene used in this experiment was the virus-associated I (VAI) RNA gene. The VAI RNA gene product defends against cellular antiviral responses by blocking the activation of the interferon-induced, double-stranded RNA-activated protein kinase PKR (Galabru J, Katze M G, Robert N, Hovanessian A G. Eur J. Biochem. 1989 Jan. 2; 178(3):581-9). The PCR amplified VAI RNA gene on a 1,724 by insert was cloned into the pCR-Blunt II-TOPO vector using a ZeroBlunt® TOPO® PCR cloning Kit (Invitrogen). Once the VAI RNA gene is introduced into mammalian cells, it is transcribed by RNA polymerase III in large amounts.

Briefly, HeLa cells were seeded into six well tissue culture plates in complete Eagle's Minimal Essential Medium (EMEM) media and incubated to >80% confluence. The next day, the number of cells in test wells from each group were counted, and 1 ml of diluted Ad35-TB.S virus or empty virus (virus that did not encode TB.S) was added to each well at a multiplicity of infection (MOI) of 50 and 100. The infection was carried out for 4 hours in a CO₂ incubator. After 4 hours, 1 ml of complete media was added to the infection mixture in each well and then the VAI gene containing pCR-Blunt II-TOPO plasmid was transiently transfected into both Ad35-TB.S virus and empty virus infected cells. Control cells containing uninfected cells were processed in the same manner. After 48 hours, cells from each well were lysed and analyzed by immunoblotting with Ag-85 specific antisera.

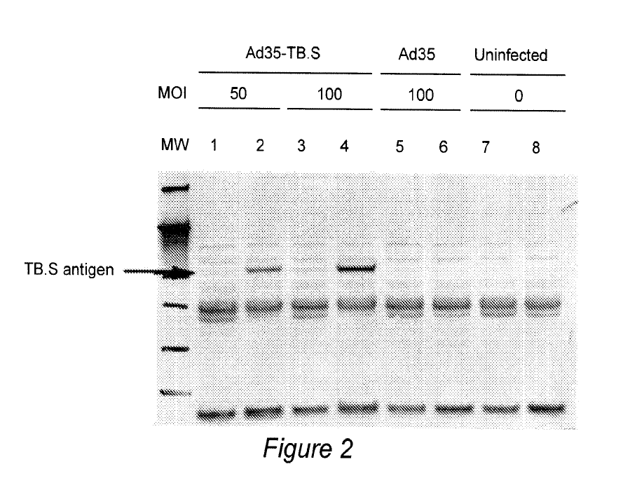

FIG. 2 shows an immunoblot comparing the production of adenovirus-expressed TB.S antigen from VAI gene transfected HeLa cells (lane 2 and 4) compared to HeLa cells that were not transfected with VAI (lanes 1 and 3). As can be seen, expression of TB antigen TB.S was significantly increased in VAI transfected cells. Higher expression of the TB.S antigen was seen at both 50 and 100 MOIs in the VAI infected cells.

This Example shows that expression of an antigen in a host cell is increased if VAI protein is also expressed in the host cell.

### Example 2

### IFN Antagonist Gene NS1 Increases the Expression of Hemagluttin (HA) Protein

An adenoviral vector vaccine construct encoding the IFN I inhibitory protein NS1 from influenza virus and the hemagluttin (HA) protein of avian influenza H₅N₁ in a bicistronic expression cassette is prepared. Expression of the HA transgene in cells infected with this adenoviral vaccine construct is compared to expression in A549 and HeLa cells infected with an analogous adenoviral vaccine construct expressing the same HA transgene but not the NS1 protein. Higher levels of HA are expressed in cells in which NS1 is also expressed. This study validates the approach of using adenovirus vectors that, in addition to encoding and expressing transgenes of interest, encode and express suppressors of the type I interferon.

### Example 3

### Enhancement of Immunogenicity of a Vaccine by the Inclusion of a Suppressor of the Type I IFN Response

To demonstrate the impact of suppression of the type I IFN response on viral vector elicited immune response, 3 groups of 10 BALB/c mice are vaccinated as follows. The first group receives only saline, 100 μl intramuscularly, the second group receives an adeno serotype 35 vector encoding a fusion of M. tuberculosis antigens 85A, 85B and RV3407 at 10e10 pfu intramuscularly, the third group receives an adeno serotype 35 vector encoding a fusion of M. tuberculosis antigens 85A, 85B and RV3407 and the VAI gene at 10e10 pfu intramuscularly. All animals are boosted with the same vaccines 2 weeks post-priming. Two weeks post boost all animals are euthanized and spleens and blood are collected.

Methods of measurement of immune and other biological responses to encoded products in animal models are well known to those skilled in the art. To measure serum IgG and IgA responses to the encoded Mtb antigens, 400-500 μA of blood is collected into individual tubes and allowed to clot by incubating for 4 hr on ice. After centrifugation in a microfuge for five minutes, the sera are transferred to fresh tubes and stored at −80° C. Mucosal IgG and IgA responses to antigens expressed by the genes of interest are determined using fecal pellets and vaginal washes that will be harvested before and at regular intervals after vaccination (Srinivasan et al., Biol. Reprod. 53: 462; 1995); (Staats et al., J. Immunol. 157: 462; 1996). Standard ELISAs are used to quantitate the IgG and IgA responses to an antigen of interest in the sera and mucosal samples (Abacioglu et al., AIDS Res. Hum. Retrovir. 10: 371; 1994); (Pincus et al., AIDS Res. Hum. Retrovir. 12: 1041; 1996). Ovalbumin can be included in each ELISA as a negative control antigen. In addition, each ELISA can include a positive control serum, fecal pellet or vaginal wash sample, as appropriate. The positive control samples are harvested from animals vaccinated intranasally with 10 μg of the antigen expressed by the gene of interest mixed with 10 μg cholera toxin, as described (Yamamoto et al., Proc. Natl. Acad. Sci. 94: 5267; 1997). The end-point titers are calculated by taking the inverse of the last serum dilution that produced an increase in the absorbance at 490 nm that is greater than the mean of the negative control row plus three standard error values.

Cellular immunity may be measured by intracellular cytokine staining (also referred to as intracellular cytokine cytometry) or by ELISPOT (Letsch A. et al., Methods 31:143-49; 2003). Both methods allow the quantitation of antigen-specific immune responses, although ICS also adds the simultaneous capacity to phenotypically characterize antigen-specific CD4+ and CD8+ T-cells. Such assays can assess the numbers of antigen-specific T cells that secrete IL-2, IL-4, IL-5, IL-6, IL-10 and IFN- (Wu et al., AIDS Res. Hum. Retrovir. 13: 1187; 1997). ELISPOT assays are conducted using commercially-available capture and detection mAbs (R&D Systems and Pharmingen), as described (Wu et al., Infect. Immun. 63:4933; 1995) and used previously (Xu-Amano et al., J. Exp. Med. 178:1309; 1993); (Okahashi et al., Infect. Immun. 64:1516; 1996). Each assay includes mitogen (Con A) and ovalbumin controls. The anti-IFN encoding vector system described herein has several advantages over delivery systems without IFN resistance genes. The antigens genes are expressed at higher levels and for longer periods of time, and therefore induce a more vigorous immune response.

This Example shows that the immune response elicited by adenoviral vector vaccines expressing both a suppressor of the type I interferon response and an immunogen of interest is increased compared to an adenoviral vector encoding only the immunogen.

### Example 4

### Enhancement of Transgene Expression from Baculovirus-Based Vaccine Vectors by the Expression of Suppressors of the Type 1 Interferon Response

A number of viral based vectors have been used to successfully transfect mammalian cells. Among those are adenovirus, adenovirus-associated virus (AAV), papovaviruses, and vacciniavirus. Adenovirus vectors have been well studied and used in a number of gene therapy trials as well as in vaccine clinical trials; although, recent negative clinical trial outcomes may restrict their use in the US (Gene Therapy, 7:110, 2000, Nature Biotechnology 26, 3-4, 2008). There also have been clinical trials using adeno-associated virus (AAV).

An alternative vector which can be used to infect mammalian cells is the insect-infecting baculovirus (Trends Biotechnol., 20, 173-180, 2002). Baculovirus is a rod virus and therefore, in contrast to capsid based viral systems, there is no limit on the amount of genetic material that can be inserted into a recombinant baculovirus. Unlike viral vectors derived from mammalian viruses, baculovirus gene expression is driven by insect specific promoters. Therefore, baculovirus genes are not expressed in human cells (Virology 125: 107-117, 1983), and thus cannot provoke an immune response. In addition, mammalian cells have no pre-existing immunity to baculovirus gene products. Further, unlike viral vectors based on mammalian viruses, no preexisting baculoviruses are within mammalian cells. Therefore, recombination of the baculovirus vector cannot occur, and infection with baculovirus cannot produce endogenous human viruses. Another advantage of the baculovirus system is that baculoviruses can be grown in large quantities in serum free culture media, which removes the potential hazard of serum contamination of the therapeutic agent with viral and prion agents.

When a mammalian promoter (e.g. CAG) or a viral internal ribosome entry site (IRES), for example encephalomyocarditis virus (EMCV) IRES, is inserted upstream of a transgene in a baculovirus, successful expression of the transgene can be achieved in mammalian cells. Baculovirus vectors would seem to be excellent candidates for vaccine development, and vaccine candidates using baculovirus systems appear to have clear advantages over most other viral vaccine systems. Unfortunately, baculovirus expression of foreign proteins in mammalian cells results in a type I interferon (IFN) response (J. Immunol., 178, 2361-2369, 2007). This IFN response limits the expression of foreign proteins by means of protein kinase R(PKR) and 2′-5′ oligoadenylate-synthetase (2′-5′ OAS). Activated PKR blocks translation by phosphorylating the subunit of eukaryotic initiation factor eIF2. On the other hand, 2-5A synthetases produce short, 2′-5′ OAS associated oligoadenylates which activate RNase L, a single-stranded specific endoribonuclease that digests mRNA and ribosomal RNA. These mechanisms likely destroy or inhibit the transcription and translation of passenger nucleic acids encoded by the baculovirus system.

Successful viral pathogens have evolved mechanisms that enable them to establish infection by blocking autocrine and paracrine responses of IFNs. Therefore, by generating a recombinant baculovirus containing a nucleic acid sequence encoding one or more proteins that interfere with host cell type I interferon (IFN) responses, significant transgene expression is observed in mammalian cells that are transfected with the recombinant baculovirus. Examples of proteins capable of modulating the type I interferon (IFN) pathway include, but are not limited to, the NSP1 protein of rotavirus, C12R protein of ectromelia virus, and NS1 of influenza. The C12R protein binds to INF-α/β thereby modulating the immune response. Recent studies have indicated that the rotavirus nonstructural protein NSP1 interacts with IRF3 and that this interaction results in the proteasome-mediated degradation of IRF3, which in turn suppresses the INF-β. The NS1 protein of influenza has been shown to have several effects on the type I IFN pathway. The activity of the carboxy-terminal domain of the NS1 protein is to inhibit the host mRNA processing mechanisms. This domain also facilitates the preferential translation of viral mRNA by direct interaction with the cellular translation initiation factor eIF4GI. In addition, by binding to dsRNA and interacting with putative cellular kinase(s), the NS1 protein prevents activation of the IFN-inducible dsRNA activated kinase (PKR), 2′,5′-oligoadenylate synthetase system, and cytokine transcription factors such as NF-KB or IRF 3 and c-Jun/ATF2. As a result, the NS1 protein inhibits the expression of INF-α and INF-β genes, thereby preventing or delaying the development of apoptosis in the infected cells, and preventing or delaying the formation of an antiviral state in neighboring cells. Thus, by constructing a baculovirus system that harbors nucleic acids encoding both an antigen and an immune response modulator, a superior vaccine candidate is generated.

The construction of recombinant baculovirus is carried out using transfer vectors. A recombinant baculovirus incorporating a foreign gene or genes of interest is produced by co-transfecting insect cells susceptible to baculovirus infection with wild type baculovirus and a transfer vector that include the gene(s) of interest. For example, U.S. Pat. No. 6,126,944 to Pellett et al., the complete contents of which is hereby incorporated by reference, describes the construction of a baculovirus transfer vector for efficient expression of foreign genes which are juxtaposed with the baculovirus polyhedrin gene at the translation initiation site, without the addition of further nucleotides to the initiation site.

The ease of construction, and capacity to accept large foreign DNA-fragments (>20 kbp), allows the development of baculoviruses having enlarged or targeted cell tropism along with more stable, temporal and cell type-specific control of transgene expression. A recombinant baculovirus encoding a fusion protein of M. tuberculosis antigens Ag85A, Ag85B, and Rv3407 is constructed. Baculoviruses have been shown to infect mammalian cells; therefore CHO, HeLa, and BHK cells are grown in tissue culture flasks are transfected with the transfer vector pcDNA3.1 encoding NS1 of influenza-A or NSP1 of rotavirus under the control of the cytomegolovirus (CMV) promoter. Control cells are infected with pcDNA3.1 vector alone. Zeomycin resistant stable transformants are expanded and seeded into 6-well tissue culture flasks in DMEM and incubated to >60% confluence. Test wells from each group are counted and cells in fresh, serum-free media are infected with the recombinant baculovirus at a multiplicity of infection (MOI) of 10, 100, 1000, and 5000 for a period of 1-2 hours. Optionally, culture media can be supplemented with 10 mM sodium butyrate to maximize transgene expression. After removal of the virus, fresh medium is added and cultures are incubated at 37° C. for 48 hours. Cultures are examined for antigen expression by immunoblotting. For Western blot analysis, cell extracts are resolved in denaturing polyacrylamide gels, and proteins are transferred to nitrocellulose membranes and immunoblotted using standard methods and Ag85-specific antisera. NS1- and NSP1-expressing cells produce transgene fusion protein(s) in excess of that observed in the control cells which do not express NS1 or NSP1.

A recombinant baculovirus which encodes both NS1 and a transgene encoding hemagluttin (HA) of avian influenza H₅N₁ in a bicistronic expression cassette is constructed. Expression of the transgene in cells infected with this recombinant baculovirus is compared to expression in a control recombinant baculovirus construct that expresses the HA transgene but not the NS1 protein. The comparison shows that significantly more HA protein is produced in cells infected with the NS1-HA recombinant, validating the approach of using a recombinant baculovirus encoding a suppressor of type I interferon. Immunogenicity studies in mice demonstrate an increase in the magnitude of the immune response to HA elicited in cells infected with recombinant baculoviruses expressing both NS1 and the HA immunogen, compared to control cells infected with recombinant baculoviruses expressing only HA immunogen.

These observations have clear and significant implications for the development and use of recombinant baculovirus vaccines. Increased transgene expression leads to improved cellular and humoral immune responses to encoded antigens. The invention thus has a broad range of applications for recombinant baculovirus vaccines encoding factors that inhibit the IFN response for the prevention and treatment of a wide variety of diseases.

### Example 5

### Use of Recombinant Viral Vectors Expressing a Factor that Inhibits the Type I IFN Response and One or More Antigens of Interest as a Vaccine

A recombinant baculovirus encoding Mtb antigens 85A, 85B and Rv3407 and the NS1 protein from influenza A is constructed, a similar vaccine lacking the NS1 protein is also constructed. To evaluate the protective efficacy of this vaccine in non-human primates, three groups of six weight and sex-matched rhesus macaques are vaccinated with 1) saline, 2) rBaculovirus AB3407 or 3) rBaculovirus AB3407NS1. Each animal in groups 2 and 3 receives 1×107 vp of the respective rBaculovirus by intramuscular injection. Fifteen weeks after vaccination, all animals are challenged by bronchial installation of approximately 300 cfu of M. tuberculosis Erdman. All animals are evaluated monthly for six months for clinical symptoms of tuberculosis by chest X-ray, weight, feeding, cough, lethargy, and immune responses to TB specific proteins. All animals that die during the six month observation period are necropsied and tissue pathology and Mtb burden by organ is measured. All moribund animals are humanely euthanized and similarly examined. Six months post-challenge all surviving animals are euthanized and necropsied for tissue pathology and Mtb burden in lungs, liver and spleen. Inclusion of NS1 in the rBaculovirus vaccine results in decreased mortality, decreased tissue damage and lower counts of viable Mtb organisms in the lungs of experimentally infected animals.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A recombinant viral vector, comprising:
one or more genetically engineered nucleic acids coding for a host cell type 1 interferon (IFN) response suppressor factor; and
one or more genetically engineered nucleic acids coding for one or more host cell active amino acid sequences;
wherein said one or more genetically engineered nucleic acids coding for said one or more host cell active amino acid sequences are over expressed in said host cell.

2. The recombinant viral vector of claim 1 wherein said host cell type 1 IFN response suppressor factor is rotavirus NSP1 or influenza virus NS1.

3. The recombinant viral vector of claim 1 wherein said host cell type 1 IFN response suppressor factor is selected from the group consisting of rotavirus NSP1, influenza virus NS1, ectromelia virus C12R protein, hepatitis C virus NS3/4A protease, vaccinia virus vIFN-α/β Rc protein, adenovirus E1A protein, C proteins of paramyxovirus, and human papillomavirus (HPV) E6 oncoprotein.

4. The recombinant viral vector of claim 1 wherein said recombinant viral vector is derived from a virus selected from the group consisting of adenoviruses, baculoviruses, pox viruses, measles viruses, polioviruses, lentiviruses, hepatitis viruses, arboviruses and vesicular stomatitis viruses.

5. The recombinant viral vector of claim 1 wherein said one or more immunostimulatory amino acid sequences are derived from one or more of rotavirus, influenza virus, ectromelia virus, hepatitis viruses, vaccinia virus, adenovirus, paramyxovirus, HPV, HIV, HTLV, enteroviruses, herpesviruses, EEE, VEE, West Nile virus, Norwalk virus, parvoviruses, dengue virus, and hemorrhagic fever virus.

6. The recombinant viral vector of claim 1, wherein said one or more host cell active amino acid sequences is an antigen.

7. The recombinant viral vector of claim 6, wherein said antigen is a Mycobacterium tuberculosis antigen.

8. A method of eliciting a tailored response in a host cell of an individual, comprising the step of
administering to said host cell of said individual a recombinant viral vector, comprising:
one or more genetically engineered nucleic acids coding for a host cell type 1 interferon (IFN) response suppressor factor; and
one or more genetically engineered nucleic acids coding for one or more host cell active amino acid sequences;
wherein said one more or more genetically engineered nucleic acids coding for said one or more host cell active amino acid sequences are over expressed by a tailored amount in said host cell, and wherein over expression of said one or more host cell active amino acid sequences in said host cell elicits said tailored response in said host cell, and
wherein said tailored amount is (a) related to said one or more genetically engineered nucleic acids coding for said host cell type 1 IFN response suppressor factor, (b) related to a promoter for said one or more genetically engineered nucleic acids coding for said host cell type 1 IFN response suppressor factor or said one or more genetically engineered nucleic acids coding for said one or more host cell active amino acids, or (c) related to a copy number of said one or more genetically engineered nucleic acids coding for said host cell type 1 IFN response suppressor factor or said one or more genetically engineered nucleic acids coding for said one or more host cell active amino acid sequences.

9. The method of claim 8, wherein said one or more host cell active amino acid sequences are immunostimulatory and said tailored response is an immune response by said individual.

10. The method of claim 8, wherein said one or more host cell active amino acid sequences are therapeutic for said host cell and said tailored response is therapeutic for said individual.

11. The method of claim 8, wherein said host cell is a cancer cell, said one or more host cell active amino acid sequences are sequences that promote apoptosis or otherwise kill cancer cells, and said tailored response is apoptosis or death of said cancer cells.

12. A method of eliciting an immune response to one or more immunogenic amino acid sequences in an individual, comprising the step of
administering to said individual a recombinant viral vector, comprising:
one or more genetically engineered nucleic acids coding for a host cell type 1 interferon (IFN) response suppressor factor; and
one or more genetically engineered nucleic acids coding said one or more immunogenic amino acid sequences;
wherein expression of said one or more immunogenic amino acid sequences from said recombinant viral vector elicits an immune response to said one or more immunogenic amino acid sequences in said individual.

13. The method of claim 12 wherein said one or more immunogenic amino acid sequences are antigens for tuberculosis or malaria.

14. A method of treating cancer in an individual, comprising the step of
administering to said individual a recombinant viral vector, comprising:
one or more genetically engineered nucleic acids coding for a host cell type 1 interferon (IFN) response suppressor factor; and
one or more genetically engineered nucleic acids coding one or more apoptosis-inducing amino acid sequences;
wherein expression of said one or more apoptosis-inducing amino acid sequences from said recombinant viral vector causes apoptosis of cancer cells in said individual.
